(19) United States
(12) Patent Application Publication
(10) Pub. No.: US 2022/0409690 A1
(43) Pub. Date: Dec. 29, 2022

(54) COMPOSITION, COMPRISING VINEGAR PICKLED EGG AS ACTIVE INGREDIENT, FOR PREVENTION, ALLEVIATION, OR TREATMENT OF DIABETIC FOOT ULCER

(71) Applicant: U Pong KANG, Seoul (KR)

(72) Inventor: U Pong KANG, Seoul (KR)

(21) Appl. No.: 17/777,595

(22) PCT Filed: Nov. 18, 2019
(86) PCT No.: PCT/KR2019/015729

§ 371 Date: May 17, 2022

(51) Int. Cl. A61K 36/8945; A61K 36/9066; A61K 36/78; A61K 36/355; A61K 36/258; A61K 36/232; A61K 35/74; A61K 35/57; A61K 9/06

(52) U.S. Cl. CPC A61K 36/8945; A61K 36/9066; A61K 36/78; A61K 36/355; A61K 36/258; A61K 36/232; A61K 35/74; A61K 35/57; A61K 9/06

(57) ABSTRACT

The present invention relates to a composition, comprising a vinegar pickled egg as an active ingredient, for prevention, alleviation, or treatment of diabetic foot ulcer. More specifically, a vinegar pickled egg fermentate obtained by adding and fermentating egg white of pullet eggs with medicinal herb vinegar does not irritate the skin and is effective for alleviating diabetic foot ulcer, thus being able to find advantageous applications as a therapeutic agent for diabetic foot ulcer, an anti-diabetic foot ulcer skin preparation for external use, or a substance for a health functional food for prevention or alleviation of diabetic foot ulcer.

### TECHNICAL FIELD

The present invention relates to a composition for preventing, alleviating or treating diabetic foot ulcers, which contains as an active ingredient an aged vinegar pickled egg product aged by adding medicinal herb vinegar to a white of a vinegar pickled egg.

### BACKGROUND ART

Diabetic foot disease refers to any problems that occur to the feet of people with diabetes, and is also called “diabetic foot.” The most typical problem that can occur to the feet of diabetic patients is foot ulcers caused by the breakage of the skin or mucosal tissues of the feet. If limited to a narrow range, diabetic foot disease can be also called an ulcer on the foot, because the ulcer thereof is the most representative lesion of diabetic foot disease.

Diabetes gradually narrows or blocks blood vessels. When the blood vessels to lower extremities are narrowed or blocked, the feet do not receive sufficient blood flow. Then, the supply of nutrients or oxygen is not smooth, and the movement and discharge of wastes become difficult, which leads to ulcers. In addition, if the sensation of the feet is dulled due to diabetic neuropathy, the feet are easy to get hurt, develop ulcers, easily deformed due to nerve abnormalities, and have weight wearing different from normal while walking, thus increasing a pressure applied in a part of the feet. In this case, the feet are put under the conditions for easily developing ulcers. Moreover, diabetes per se delays wound healing. Thus, upon the occurrence of a traumatic lesion to the feet, self-healing becomes slow, thus making it easy to develop ulcers and delay healing.

About 47% of all foot diseases are diabetic foot disease, and about 15% of diabetic patients have foot ulcers at least once in their lifetime, and about 1 to 3% of the patients undergo a surgery of having a part of their legs amputated. Foot ulcers caused by diabetes often recur once they occur, with about 30% of patients experiencing a recurrence within one year, and more than half of those who have undergone a surgery for diabetic foot ulcers also have a surgery on the other leg within four years after surgery. In particular, it is said that about 40% of the causes of hospitalization of diabetic patients is due to diabetic foot disease. In addition, it is known that more than half of the causes of partial amputation of a leg, except for cases due to trauma, are diabetic foot ulcers. As such, diabetic foot ulcer is a very important cause of lowering the quality of life of diabetic patients.

The goals of treating foot ulcers are: first, to prevent the ulcer, second, to minimize the progression to infection and necrosis by treating the ulcer at an early stage of development, and third, not to amputate if possible or minimize amputation even if infection and necrosis occur, thereby providing ultimate treatment for well-functioning feet.

A traditional approach to treat foot ulcers include desloughing, debridement, pressure relief (e.g., rest, special footwear, shoe inserts and castings), antibiotic treatment of infections, and wound dressings.

The present inventors have tried to provide a safe composition of natural raw materials that can improve a therapeutic effect by early treatment of diabetic foot ulcers.

Meanwhile, a vinegar pickled egg is a food made by immersing an egg in vinegar and melting a shell. When an egg is soaked in vinegar for five or six days, a shell thereof will dissolve in the vinegar and become soft. An egg white becomes hydrated and swells, and an egg yellow becomes hard like a semi-boiled egg, which is called a vinegar pickled egg. A reason for which an eggshell melts and becomes soft is that the vinegar's main ingredient, acetic acid, dissolves lime powder. The hardening of yolk of an egg is involved in an acid coagulation phenomenon, and vinegar has an effect of hardening proteins well.

An egg contains an ingredient called antitrypsin, which inhibits protein digestion, and thus can put a burden on infants, the elderly, or patients with digestive system with weak digestion. In addition, avidin contained in the egg white interferes with an action of biotin, one of the vitamins, and may cause dermatitis or hair loss. The vinegar pickled egg may be a food that solves all of these problems with eggs by the action of vinegar.

The vinegar pickled egg is good for digestion and absorption, and is effective in returning weakened physical strength to normal, and its scope of utility is large as it helps to facilitate metabolic functions. Hippocrates, the founder of medicine, also points out in his book that the vinegar pickled egg is good for patients who are in a recovery stage. Judging from this, it can be said that the efficacy of the vinegar pickled egg has been recognized for 2,000 years ago. In addition, the vinegar pickled egg is known to be effective in preventing high blood pressure, arteriosclerosis, gastroptosis, hepatitis, and diabetes (when taken for a long time).

The most common way of consuming the vinegar pickled egg is to eat as it is, which is obtained by being soaked in vinegar, that is, to eat the vinegar pickled egg with the soft skin as it is or to eat a mixed form of yolk and white while discarding the hard skin. A cosmetic composition using the vinegar pickled egg has been developed by the present inventors, but there is no research on a therapeutic agent of diabetic foot ulcers using the vinegar pickled egg.

Meanwhile, Korea Registered Patent No. 1063875 discloses a cosmetic composition for skin care containing a vinegar pickled egg as an active ingredient and a method for preparing the same, and Korean Registered Patent No. 1534468 discloses a pharmaceutical composition for treating diabetic ulcers, including edible beeswax; and sesame oil extracts of Scutellaria baicalensis, coptis root, phellodendron bark, earthworm, and poppy capsules, but there has been no disclosure about a composition for preventing, alleviating or treating diabetic foot ulcers, containing an aged vinegar pickled egg product aged by adding medicinal herb vinegar to an white of a vinegar pickled egg, as an active ingredient.

### DISCLOSURE

### Technical Problem

The present invention has been derived from the above needs, and it has been confirmed that an aged vinegar pickled egg product aged by adding medicinal herb vinegar to a white of a vinegar pickled egg, alleviates diabetic foot ulcers without irritating the skin, thereby completing the present invention.

### Technical Solution

To solve the above problems, the present invention may provide a pharmaceutical composition for preventing or treating diabetic foot ulcers, which contains as an active ingredient an aged vinegar pickled egg product aged by adding medicinal herb vinegar to a white of a vinegar pickled egg.

In addition, the present invention may provide a skin preparation for external use for preventing or alleviating diabetic foot ulcers, which contains as an active ingredient an aged vinegar pickled egg product aged by adding medicinal herb vinegar to a white of a vinegar pickled egg.

Furthermore, the present invention may provide a health functional food composition for preventing or alleviating diabetic foot ulcers, which contains as an active ingredient an aged vinegar pickled egg product aged by adding medicinal herb vinegar to a white of a vinegar pickled egg.

### Advantageous Effects

The present invention relates to a composition for preventing, alleviating or treating diabetic foot ulcers, containing a vinegar pickled egg as an active ingredient, in which an aged vinegar pickled egg product aged by adding medicinal herb vinegar to a white of a vinegar pickled egg, does not irritate the skin and is effective for alleviating diabetic foot ulcers, thus being able to find advantageous applications as a pharmaceutical composition for preventing or treating diabetic foot ulcers, a skin preparation for external use for preventing or alleviating diabetic foot ulcers, or a substance for a health functional food for preventing or alleviating diabetic foot ulcers.

### DESCRIPTION OF DRAWINGS

FIG. 1 is a picture of confirming an alleviation effect by applying a composition of the present invention to an affected area of a patient with diabetic foot ulcers (male, 78 years old).

FIG. 2 is a picture of confirming an alleviation effect by applying the composition of the present invention to an affected area of a patient with diabetic foot ulcer (male, 62 years old).

FIG. 3 is a picture of confirming an alleviation effect by applying the composition of the present invention to an affected area of a patient with diabetic foot ulcer (female, 64 years old).

FIG. 4 shows the results of confirming blood sugar of an animal model with streptozotocin-induced diabetes as measured (a) before drug treatment and (b) after drug treatment. * indicates that the blood sugar of a diabetic group was significantly increased compared to a normal group, and p<0.05.

FIG. 5 is a picture of showing a resulting change in a wound site according to the course of drug treatment.

FIG. 6 shows the results of schematizing a size of a wound site, and (a) indicates a change in a size of a wound site according to an experimental period and (b) indicates a comparison of a daily change in a size of a wound site for each group compared to each drug treatment group. * indicates that a size of a wound site in the diabetic group was significantly increased compared to the normal group, and p<0.05. # indicates that a size of a wound site in a group treated with Madecassol or Experimental Example 2 was significantly increased compared to the diabetic group, and p<0.05.

### BEST MODE

The present invention relates to a pharmaceutical composition for preventing or treating diabetic foot ulcers, which contains as an active ingredient an aged vinegar pickled egg product aged by adding medicinal herb vinegar to a white of a vinegar pickled egg.

In the pharmaceutical composition of the present invention, a white of the vinegar pickled egg and medicinal herb vinegar may be mixed at a weight ratio of 0.8 to 1.2:0.8 to 1.2, more preferably at a weight ratio of 1:1, but is not limited thereto.

In one embodiment of the present invention, a white of the vinegar pickled egg may be prepared by:

(a) washing a surface of an egg, followed by drying;

(b) immersing the washed and dried egg in step (a) in a mixture of persimmon vinegar and Benicasa hispida vinegar for six to eight days, followed by taking out, and washing the surface of the egg with a mixture of unused persimmon vinegar and Benicasa hispida vinegar;

(c) repeating one to three times the process of immersing the washed egg in step (b) in medicinal herb vinegar for six to eight days to prepare a vinegar pickled egg, removing the medicinal herb vinegar therefrom, and washing the surface of the vinegar pickled egg with unused fresh medicinal herb vinegar; and

(d) separating the white from the vinegar pickled egg after step (c),

more preferably by:

(a) washing a surface of an egg, followed by drying;

(b) immersing the washed and dried egg in step (a) in a mixture of persimmon vinegar and Benicasa hispida vinegar at a weight ratio of 0.8 to 1.2:0.8 to 1.2 for six to eight days, followed by taking out, and washing the surface of the egg with a mixture of unused persimmon vinegar and Benicasa hispida vinegar;

(c) repeating one to three times the process of immersing the washed egg in step (b) in medicinal herb vinegar for six to eight days to prepare a vinegar pickled egg, removing the medicinal herb vinegar therefrom, and washing the surface of the vinegar pickled egg with unused fresh medicinal herb vinegar; and

(d) separating the white from the vinegar pickled egg after step (c), and

much more preferably by:

(a) washing a surface of an egg, followed by drying;

(b) immersing the washed and dried egg in step (a) in a mixture of persimmon vinegar and Benicasa hispida vinegar at a weight ratio of 1:1 for seven days, followed by taking out, and washing the surface of the egg with a mixture of unused persimmon vinegar and Benicasa hispida vinegar;

(c) repeating twice the process of immersing the washed egg in step (b) in medicinal herb vinegar for seven days to prepare a vinegar pickled egg, removing the medicinal herb vinegar therefrom, and washing the surface of the vinegar pickled egg with unused fresh medicinal herb vinegar; and

(d) separating the white from the vinegar pickled egg after step (c), but is not limited thereto.

In the method for preparing the vinegar pickled egg, the persimmon vinegar and Benicasa hispida vinegar in step (b) may be a vinegar prepared by fermenting persimmon or Benicasa hispida (pumpkin), which is a raw material for each vinegar, with acetic acid for at least one year, but is not limited thereto.

In one embodiment of the present invention, the medicinal herb vinegar may be a vinegar prepared by mixing Chinese dioscorea, arrowroot, Dendropanax morbifera, Huttuynia cordata, Saururus chinensis, Lonicera japonica, balloon flower, Cassia obtusifolia, ginseng, adlay, Ulmus macrocarpa, Liriope platyphylla, licorice, Astragalus membranaceus, Angelica gigas, kelp, Achyranthes japonica, Rubus coreanus, Eriobotrya japonica, Acer mono sap and brown sugar, and fermenting the mixture by adding acetic acid bacteria, but is not limited thereto.

In one embodiment of the present invention, the aged vinegar pickled egg product aged by adding medicinal herb vinegar to a white of a vinegar pickled egg, may be prepared by:

(a) washing a surface of an egg, followed by drying;

(b) immersing the washed and dried egg in step (a) in a mixture of persimmon vinegar and Benicasa hispida vinegar for six to eight days, followed by taking out, and washing the surface of the egg with a mixture of unused persimmon vinegar and Benicasa hispida vinegar;

(c) repeating one to three times the process of immersing the washed egg in step (b) for six to eight days in the medicinal herb vinegar obtained by mixing Chinese dioscorea, arrowroot, Dendropanax morbifera, Huttuynia cordata, Saururus chinensis, Lonicera japonica, balloon flower, Cassia obtusifolia, ginseng, adlay, Ulmus macrocarpa, Liriope platyphylla, licorice, Astragalus membranaceus, Angelica gigas, kelp, Achyranthes japonica, Rubus coreanus, Eriobotrya japonica, Acer mono sap and brown sugar, fermenting the mixture by adding acetic acid bacteria to prepare a vinegar pickled egg, removing the medicinal herb vinegar therefrom, and washing the surface of the vinegar pickled egg with unused fresh medicinal herb vinegar;

(d) separating the white from the vinegar pickled egg after step (c); and

(e) adding the medicinal herb vinegar in step (c) to the white of the vinegar pickled egg in step (d), followed by aging,

preferably by:

(a) washing a surface of an egg, followed by drying;

(b) immersing the washed and dried egg in step (a) in a mixture of persimmon vinegar and Benicasa hispida vinegar at a weight ratio of 0.8 to 1.2:0.8 to 1.2 for six to eight days, followed by taking out, and washing the surface of the egg with a mixture of unused persimmon vinegar and Benicasa hispida vinegar;

(c) repeating one to three times the process of immersing the washed egg in step (b) for six to eight days in the medicinal herb vinegar obtained by mixing Chinese dioscorea, arrowroot, Dendropanax morbifera, Huttuynia cordata, Saururus chinensis, Lonicera japonica, balloon flower, Cassia obtusifolia, ginseng, adlay, Ulmus macrocarpa, Liriope platyphylla, licorice, Astragalus membranaceus, Angelica gigas, kelp, Achyranthes japonica, Rubus coreanus, Eriobotrya japonica, Acer mono sap and brown sugar, fermenting the mixture by adding acetic acid bacteria to prepare a vinegar pickled egg, removing the medicinal herb vinegar therefrom, and washing the surface of the vinegar pickled egg with unused fresh medicinal herb vinegar;

(d) separating the white from the vinegar pickled egg after step (c); and

(e) adding the medicinal herb vinegar in step (c) to the white of the vinegar pickled egg in step (d) at a weight ratio of 0.8 to 1.2:0.8 to 1.2, followed by aging for three to four days,

much more preferably by:

(a) washing a surface of an egg, followed by drying;

(b) immersing the washed and dried egg in step (a) in a mixture of persimmon vinegar and Benicasa hispida vinegar at a weight ratio of 1:1 for seven days, followed by taking out, and washing the surface of the egg with a mixture of unused persimmon vinegar and Benicasa hispida vinegar;

(c) repeating twice the process of immersing the washed egg in step (b) for seven days in the medicinal herb vinegar obtained by mixing Chinese dioscorea, arrowroot, Dendropanax morbifera, Huttuynia cordata, Saururus chinensis, Lonicera japonica, balloon flower, Cassia obtusifolia, ginseng, adlay, Ulmus macrocarpa, Liriope platyphylla, licorice, Astragalus membranaceus, Angelica gigas, kelp, Achyranthes japonica, Rubus coreanus, Eriobotrya japonica, Acer mono sap and brown sugar, fermenting the mixture by adding acetic acid bacteria to prepare a vinegar pickled egg, removing the medicinal herb vinegar therefrom, and washing the surface of the vinegar pickled egg with unused fresh medicinal herb vinegar;

(d) separating the white from the vinegar pickled egg after step (c); and

(e) adding the medicinal herb vinegar in step (c) to the white of the vinegar pickled egg in step (d) at a weight ratio of 1:1, followed by aging for three to four days, but is not limited thereto.

A pharmaceutical composition of the present invention may include a pharmaceutically acceptable carrier in addition to an active ingredient, and the carrier may be one conventionally used in formulating a preparation, including, but not limited thereto, lactose, dextrose, sucrose, sorbitol, mannitol, starch, acacia rubber, calcium phosphate, alginate, gelatin, calcium silicate, microcrystalline cellulose, polyvinyl pyrrolidone, cellulose, water, syrup, methyl cellulose, methyl hydroxybenzoate, propylhydroxybenzoate, talc, magnesium stearate, mineral oil and the like. The pharmaceutical composition of the present invention may further include lubricant, humectant, a sweetening agent, a flavoring agent, emulsifier, a suspending agent, preservative, etc. in addition to the above ingredients.

A suitable dosage of the pharmaceutical composition according to the present invention may be variously prescribed depending on factors such as a method for formulating into preparation, an administration method, a patient's age, weight, gender, pathological condition, food, administration time, administration route, excretion rate and reaction sensitivity.

The pharmaceutical composition of the present invention may be orally or parenterally administered, in which the parenteral administration may be performed through local application to the skin, intravenous injection, subcutaneous injection, intramuscular injection, intraperitoneal injection, transdermal administration, etc. Considering that the pharmaceutical composition of the present invention is applied to treat or prevent diabetic foot ulcers, it is preferable that the composition is locally applied to the skin.

A concentration of the active ingredient contained in the composition of the present invention may be determined in consideration of a therapeutic purpose, a patient's condition, a required period, etc., and is not limited to a concentration within a specific range.

The pharmaceutical composition of the present invention may be formulated into preparations by using pharmaceutically acceptable carriers or excipients according to a method easily practicable by those skilled in the art to which the present invention pertains, and thus may be prepared in a unit dose form or prepared by being inserted into a multi-dose container. In this case, the pharmaceutical composition may be prepared into any one formulation selected from an injectable agent, cream, patch, spray, ointment, plaster, lotion, liniment, paste and cataplasma, and may further include a dispersing agent or a stabilizing agent.

In addition, the present invention relates to a skin preparation for external use for preventing or alleviating diabetic foot ulcers, which contains as an active ingredient an aged vinegar pickled egg product aged by adding medicinal herb vinegar to a white of a vinegar pickled egg.

The skin preparation for external use according to the present invention may be used by directly applying to an affected area, and may be prepared by further mixing natural oil, fatty acid, starch, etc., as a suitable carrier for transdermal administration, if necessary, but is not limited thereto.

In one embodiment of the present invention, the skin preparation for external use may be prepared into a formulation of power for external use, tablet for external use, liquid for external use, ointment, cream, gel, plaster, dressing, patch or spray, but is not limited thereto.

In addition, the present invention relates to a health functional food composition for preventing or alleviating diabetic foot ulcers, which contains as an active ingredient an aged vinegar pickled egg product aged by adding medicinal herb vinegar to a white of a vinegar pickled egg.

The health functional food composition may be prepared in the form of powder, granule, pill, tablet, capsule, candy, syrup or beverage, but is not limited thereto.

If the health functional food composition of the present invention is used as food additives, the health functional food composition may be added as it is, or may be used along with other foods or food ingredients, and may be appropriately used according to a conventional method. An amount of the active ingredient may be appropriately used depending on a purpose of use thereof (prevention or alleviation). In general, when preparing food or beverage, the health functional food composition of the present invention may be added in an amount of 15 parts by weight or less, preferably 10 parts by weight or less, based on a raw material. However, in the case of long-term intake for health purposes, the amount may be equal to or less than the above range and the active ingredient may be used in an amount equal to or greater than the above range since there is no problem in terms of safety.

A type of the health functional food is not particularly limited. An example of food, to which the health functional food composition may be added, may include meats, sausages, breads, chocolates, candies, snacks, confectioneries, pizzas, instant noodles, other noodles, chewing gums, dairy products including ice creams, various types of soup, beverages, teas, health drinks, alcohol beverages, vitamin complexes and the like, and include all the health foods in a conventional sense.

In addition, the health functional food composition of the present invention may be prepared as a food, in particular, a functional food. The functional food of the present invention may include ingredients commonly added upon the preparation of foods, and may include, for example, proteins, carbohydrates, fats, nutrients and seasonings. For example, when prepared as a health drink, natural carbohydrates or flavoring agents may be included as additional ingredients in addition to the active ingredient. The natural carbohydrates may be preferably monosaccharides (e.g., glucose, fructose, etc.), disaccharides (e.g., maltose, sucrose, etc.), oligosaccharides, polysaccharides (e.g., dextrin, cyclodextrin, etc.) or sugar alcohols (e.g., xylitol, sorbitol, erythritol, etc.). The flavoring agent used herein may include natural flavoring agents (e.g., thaumatin, stevia extract, etc.) and synthetic flavoring agents (e.g., saccharin, aspartame, etc.).

In addition to the above, the health functional food composition may further contain various nutritional supplements, vitamin, electrolyte, flavoring agent, coloring agent, pectic acid and salts thereof, alginic acid and salts thereof, organic acid, protective colloidal thickener, pH adjusting agent, stabilizer, preservative, glycerin, alcohol, carbonator used in carbonated beverage, etc.

Hereinafter, the present invention will be described in more detail through the following preparation examples and exemplary embodiments. These preparation examples and exemplary embodiments are provided only for the purpose of illustrating the present invention in more detail, and thus it will be apparent to those skilled in the art that the scope of the present invention is not limited thereto.

### Preparation Example 1. Preparation of Medicinal Herb Vinegar

A medicinal herb vinegar was prepared by mixing each 10 kg of Chinese dioscorea, arrowroot, Dendropanax morbifera, Huttuynia cordata, Saururus chinensis, Lonicera japonica, balloon flower, Cassia obtusifolia, ginseng, adlay, Ulmus macrocarpa, Liriope platyphylla, licorice, Astragalus membranaceus, Angelica gigas, kelp, Achyranthes japonica, Rubus coreanus, Eriobotrya japonica, Acer mono sap and brown sugar, and fermenting the mixture by adding acetic acid bacteria.

### Preparation Example 2. Preparation of Aged Vinegar Pickled Egg Product Mixed with Vinegar Pickled Egg and Medicinal Herb Vinegar

A surface of an egg was washed to clearly remove foreign materials therefrom, and then the surface of the egg was dried. After that, the dried egg was immersed for seven days in a mixture of persimmon vinegar and Benicasa hispida vinegar at a weight ratio of 1:1 and taken out to wash the surface of the egg with a mixture of unused persimmon vinegar and Benicasa hispida vinegar.

The washed egg with the persimmon vinegar and Benicasa hispida vinegar was immersed in the medicinal herb vinegar prepared in preparation example 1 for seven days to prepare a vinegar pickled egg, remove the medicinal herb vinegar therefrom, and wash the surface of the vinegar pickled egg with unused fresh medicinal herb vinegar, and then the process of immersing and washing the egg with medicinal herb vinegar was repeated twice.

After that, only a white was separated from the prepared vinegar pickled egg, and the medicinal herb vinegar of above preparation example 1 was added to the separated white of the vinegar pickled egg at a weight ratio of 1:1, and aged for three to four days.

### Example 1: Skin Irritation Test

A skin patch test was performed to carry out a skin irritation test for an aged vinegar pickled egg product (hereinafter, Experimental Example 1) of the present invention, which was prepared in Preparation Example 2, an aged vinegar pickled egg product (hereinafter, Comparative Example 2) prepared through the method of Preparation Example 2, except using persimmon vinegar instead of medicinal herb vinegar, and an aged product (hereinafter, Comparative Example 1) obtained by adding medicinal herb vinegar to a white of a vinegar pickled egg, which was not prepared from a vinegar pickled egg, respectively. Subjects were selected from among those aged 15-30 years old without a skin disease at a test site and the test was conducted on each of 15 subjects. After wiping an upper arm, which is a patch site, with 70% (v/v) ethanol, followed by drying, 0.1 g of each sample was added and applied to the upper arm of the human body for 24 hours. After removing the patch, a primary skin reaction was checked within one hour. In 48 hours after removing the patch, the test was performed again and the results thereof are shown in table 1. A skin reaction was determined according to the following test criteria.

TABLE 1
Rating results of skin irritation test (n = number of subjects)

| | | Rating Results | | | | |
|---|---|---|---|---|---|---|
| Prescription | Number of subjects | − | ± | + | ++ | +++ |
| Comparative Example 1 | 15 | 13 | 2 | 0 | 0 | 0 |
| Comparative Example 2 | 15 | 7 | 4 | 3 | 1 | 0 |
| Experimental Example 1 | 15 | 15 | 0 | 0 | 0 | 0 |

* Skin reaction test criteria
−: No response
±: Insignificant positive response (erythema)
+: Positive response (erythema)
++: Strong positive response (erythema, erythema)
+++: Severe positive response (erythema, erythema, blister)

As a result, as shown in above table 1, it could be seen that Experimental Example 1 prepared by the preparation method of the present invention does not show any skin irritation, but Comparative Example 1 using an egg white shows an insignificant erythema response in about 13% of the subjects, and Comparative Example 2 aged with persimmon vinegar shows skin irritation accompanied by erythema on the skin in more than half of the subjects.

### Example 2. Effect of Alleviating Diabetic Foot Ulcers

For a total of 30 patients suffering from severe diabetes mellitus and showing foot ulcers, Experimental Example 1 of the present invention or Comparative Example 1, which showed almost no skin erythema response in Example 1, was prepared in the form of an ointment and then applied to an affected area to confirm an effect on the alleviation of diabetic foot ulcers.

Patients promised to quit smoking and not to drink alcohol during a trial period. For 15 patients of group 1, above Experimental Example 1 (an aged vinegar pickled egg product aged by adding medicinal herb vinegar to a white of a vinegar pickled egg) was prepared in the form of an ointment (hereinafter, Experimental Example 2) according to a conventional method and applied. For 15 patients of group 2, above Comparative Example 1 (an aged product aged by adding medicinal herb vinegar to a white of an egg, which was not prepared as a vinegar pickled egg) was prepared in the form of an ointment (Comparative Example 3) by the same method as in Experimental Example 2 and applied.

Experimental Example 2 or Comparative Example 3 was applied to an affected area once or twice a day after wiping the affected area of a patient with foot ulcers with physiological saline for up to 60 days, and the appearance of foot ulcers before and after application was observed.

As a result, as shown in table 2, it was confirmed that the application of the ointment of Experimental Example 2 of the present invention has an excellent effect of having most of pains disappear and alleviating foot ulcers compared to the case of applying Comparative Example 3.

TABLE 2
Effect of alleviating foot ulcers by application
of the composition of the present invention
(n = number of subjects)

| | Much | Slightly | No | |
|---|---|---|---|---|
| | alleviated | alleviated | change | Aggravated |
| Comparative | 0 | 2 | 12 | 1 |
| Example 3 | | | | |
| Experimental | 10 | 3 | 2 | 0 |
| Example 2 | | | | |

Hereinafter, three representative cases among the subjects who showed an alleviation effect will be described.

<Alleviation Case 1>

Subject: Male, 78 years old

Treatment period: Jul. 3, 2017. to Jul. 30, 2017

Treatment method: The affected area was wiped with physiological saline and the ointment of Experimental Example 2 of the present invention was applied once or twice a day.

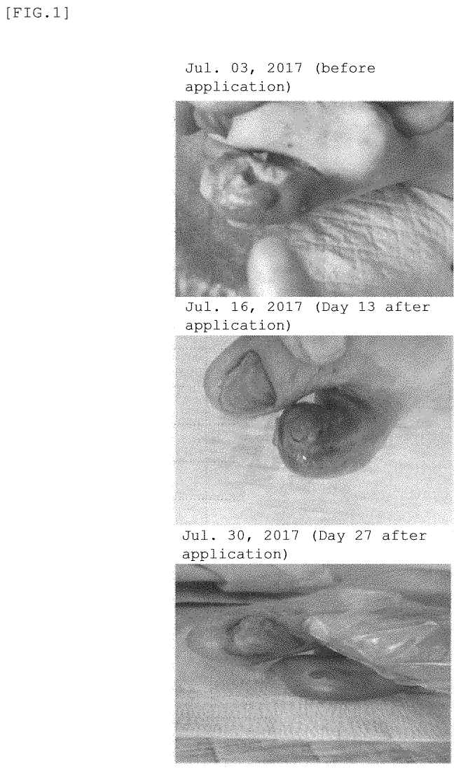

Treatment results: As shown in FIG. 1, it could be confirmed that the affected area of the patient with diabetic foot ulcers is remarkably alleviated on the 27th day after application of the ointment of Experimental Example 2 of the present invention.

<Alleviation Case 2>

Subject: Male, 62 years old

Treatment period: May 10, 2017 to Jul. 1, 2017

Treatment method: The affected area was wiped with physiological saline and the ointment of Experimental Example 2 of the present invention was applied once or twice a day.

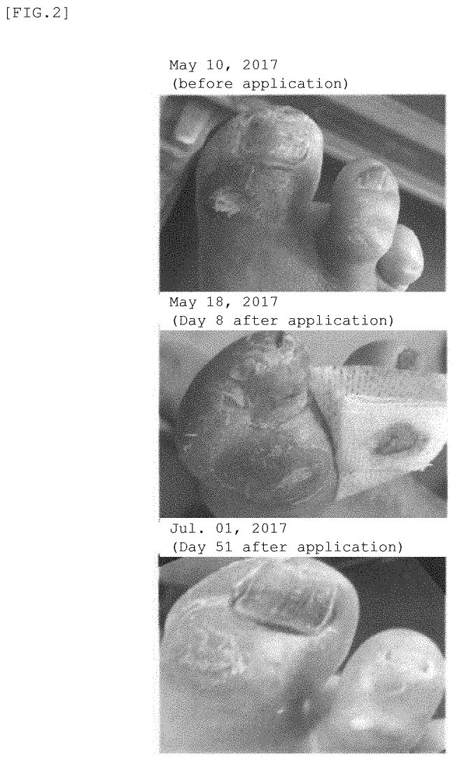

Treatment results: A treatment effect was delayed because the affected area was covered with gauze for the livelihood of the subject wearing socks and walkers, but as shown in FIG. 2, it could be confirmed that the patient's affected area was remarkably alleviated on the 51st day after application of the ointment of Experimental Example 2.

<Alleviation Case 3>

Subject: Female, 64 years old

Treatment period: Nov. 25, 2017 to Dec. 7, 2017

Treatment method: The affected area was wiped with physiological saline and the ointment of Experimental Example 2 of the present invention was applied once or twice a day.

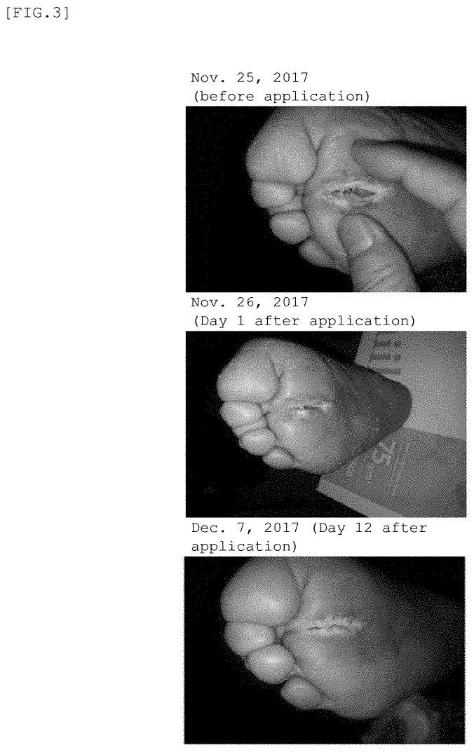

Treatment results: As shown in FIG. 3, it could be confirmed that the affected area of the patient is remarkably alleviated on the 12th day after application of the ointment of Experimental Example 2 of the present invention.

### Example 3. Effect of Alleviating Delayed Wound Healing in Diabetic Animal Model

An animal model with streptozotocin-induced diabetes was induced to have a wound and treated with the ointment of Experimental Example 2, which had shown an excellent effect of alleviating diabetic foot ulcers, so as to confirm if there occurs the effect of alleviating a delayed wound healing, which is a similar clinical symptom in diabetic foot ulcers.

1) Materials and Methods

Test Drug

The ointment of Experimental Example 2 was stored at room temperature, and 50% (w/w) of Experimental Example 2 was prepared by mixing with 1% (w/w) of carbopol hydrogel (sigma, USA) as an excipient. As a control drug, Madecassol (Dongkook Pharmaceutical Co., Ltd., Korea) was used.

Breeding of Laboratory Animals and Design of Experiments

Six-week-old male SD rats (Coatech, Korea) were used after acclimatization for one week. Feed and drinking water were provided ad libitum. In one week later, the rats were allowed to fast for 16 hours, and then streptozotocin was diluted in citrate buffer (pH 3.0) and intraperitoneally administered at a concentration of 60 mg/kg to induce diabetes. Normal animals were administered with only the citrate buffer in the same amount.

In one week later, blood was collected from a caudal vein to measure blood sugar, and only individuals with a blood sugar of 300 mg/dL or higher were selected and randomly divided into groups as shown in table 3 below so that an average blood sugar per group is the same.

TABLE 3

| | | | Applied | Number |
|---|---|---|---|---|
| | | Administered | amount | of |
| Test group | | substance | (μL/skin) | animals |
| G1 | Normal group (NOR) | Carbopol hydrogel | 50 | 9 |
| G2 | Diabetic group (DM) | Carbopol hydrogel | 50 | 9 |
| G3 | Diabetes + Madecassol | Madecassol | 50 | 9 |
| | | ointment | | |
| G4 | Diabetes + 50% (w/w) | 50% (w/w) | 50 | 9 |
| | Experimental Example | Experimental | | |
| | 2 | Example 2 | | |
| G5 | Diabetes + Undiluted | Undiluted solution | 50 | 9 |
| | solution of | of Experimental | | |
| | Experimental Example | Example 2 | | |
| | 2 | | | |

In order to induce a similar environment in which foot ulcers occur, animals were induced to have an ulcerative wound on the skin after grouping.

After anesthetizing each of experimental animals with isoflurane (Foran, JW Pharmaceutical), the hair on the skin of the back was removed with an electric clipper, clearly shaved with a razor blade, and disinfected with 70% (v/v) ethanol. A full-thickness excision wound was made at a distance of 1.5 cm outwardly from the lumbar spine by using an 8 mm biopsy punch (Biopsy punch, Ziefel, Germanay). After excision, sutures were not performed to induce secondary healing. From the day of inducing the wound, the substance to be administered as shown in table 3 was applied to the wound site once a day.

Evaluation of Wound Closure Rate

A picture was taken of the wound site at the same time slot at intervals of three days from immediately after inducing the wound to immediately after the final drug treatment, and a diameter of the circular wound, which was observed with the naked eye, was measured with calipers (Mitutoyo Co., Kawasaki, Japan) capable of measuring the length in units of 0.05 mm. An autopsy was performed about two weeks after application of the drug, and the skin at the wound site was excised and fixed in 10% (v/v) neutralized formalin and stored.

2) Results

Diabetes Induction and Change in Blood Sugar

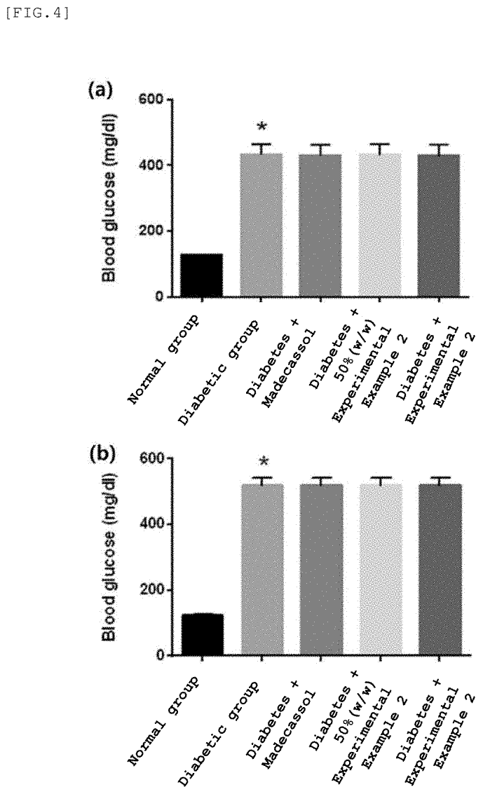

After measuring the blood sugar of animal models with streptozotocin-induced diabetes, the animals were divided into groups so that an average blood sugar for each group was about 430 mg/dl as shown in FIG. 4(a). As a result of measuring blood sugar after treatment with streptozotocin and drug for two weeks thereafter, the average blood sugar was about 510 mg/dl as shown in FIG. 4(b), thus confirming that the application of the drug to the wound has no effect on blood sugar.

Analysis of the Effect of Promoting Wound Healing

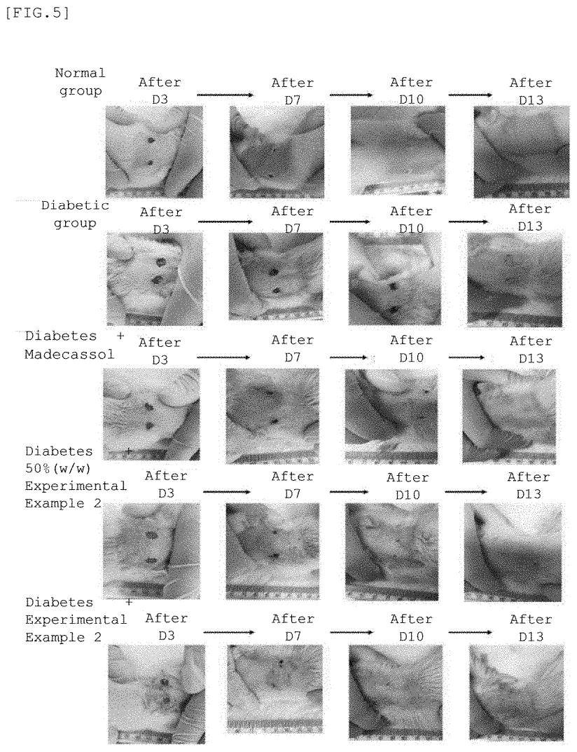

After inducing a wound through 8 mm full-thickness excision in an animal with induced diabetes, the drug corresponding to each group was applied to the affected area once a day for two weeks, and the wound site was photographed at intervals of three or four days. As a result, as shown in FIG. 5, it was confirmed that the wound healing effect delayed by diabetes is improved when the ointment of Experimental Example 2 is applied.

In the wound area, a size of the affected area was decreased over the course of three to 13 days, and in the case of the normal group, in particular, all wounds were cured 13 days after induction of the wound without any trace of the wound. In contrast, in the case of the diabetic group, the size of the affected area was not greatly decreased even after seven days, and the wound healing was not completed even after 13 days, so it was possible to visually see the affected area. However, it could be confirmed that the wound site of the animal treated with Experimental Example 2 becomes rapidly smaller in size compared to the diabetic group.

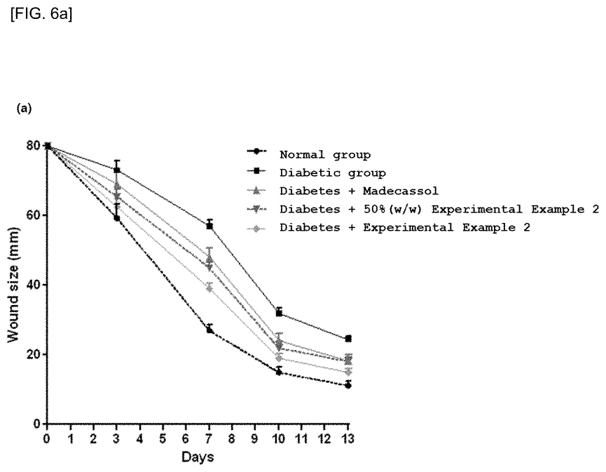

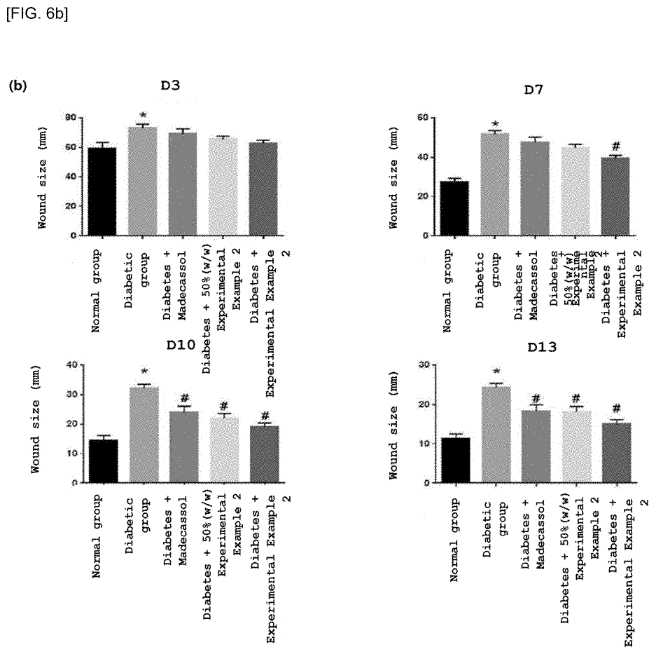

In addition, for a more accurate analysis, the size of the wound site was measured and quantitatively analyzed. As a result, as shown in FIG. 6, the size of the wound in the normal group was rapidly decreased, whereas in the case of the diabetic group, the size of the affected area was larger than that of the normal group at all time points of measurement, and even after 13 days, the size of the wound in the diabetic group was maintained to be at least twice as large as that of the normal group.

In contrast, in the case of the group treated with Experimental Example 2, a wound healing rate was faster than that of the diabetic group, and the size of the wound site was reduced by about 40% compared to the diabetic group at the time of 13 days.

1. A pharmaceutical composition for preventing or treating diabetic foot ulcers, comprising as an active ingredient an aged vinegar pickled egg product aged by adding medicinal herb vinegar to a white of a vinegar pickled egg.

2. The pharmaceutical composition of claim 1, wherein the medicinal herb vinegar is prepared by mixing Chinese dioscorea, arrowroot, Dendropanax morbifera, Huttuynia cordata, Saururus chinensis, Lonicera japonica, balloon flower, Cassia obtusifolia, ginseng, adlay, Ulmus macrocarpa, Liriope platyphylla, licorice, Astragalus membranaceus, Angelica gigas, kelp, Achyranthes japonica, Rubus coreanus, Eriobotrya japonica, Acer mono sap and brown sugar, and fermenting the mixture by adding acetic acid bacteria.

3. A skin preparation for external use for preventing or alleviating diabetic foot ulcers, comprising as an active ingredient an aged vinegar pickled egg product aged by adding medicinal herb vinegar to a white of a vinegar pickled egg.

4. The skin preparation for external use of claim 3, wherein the skin preparation for external use is powers for external use, tablets for external use, liquids for external use, ointments, creams, gels, plasters, dressings, patches or sprays.

5. A health functional food composition for preventing or alleviating diabetic foot ulcers, comprising as an active ingredient an aged vinegar pickled egg product aged by adding medicinal herb vinegar to a white of a vinegar pickled egg.
